(19) United States
(12) Patent Application Publication
(10) Pub. No.: US 2003/0180803 A1
(43) Pub. Date: Sep. 25, 2003

(54) Lead molecule generation

(76) Inventors: Ah Wing Edith Chan, London (GB); Roman Aleksander Laskowski, London (GB)

Correspondence Address: ARENT FOX KINTNER PLOTKIN & KAHN, 1050 CONNECTICUT AVENUE, N.W., SUITE 400, WASHINGTON, DC, 20036, US

(21) Appl. No.: 10/275,953

(22) PCT Filed: May 16, 2001
(86) PCT No.: PCT/GB01/02177

(30) Foreign Application Priority Data
May 16, 2000 (GB) 0011818.2

(51) Int. Cl. G01N 33/53; G06G 7/48; G06G 7/58

(52) U.S. Cl. 435/7.1; 703/11

(57) ABSTRACT

The invention concerns a method for generating lead molecules capable of interacting with a target protein of interest. In particular, the method identifies the binding sites of proteins, characterises the types of atomic interactions available within those binding sites, and uses this information as a means of identifying lead molecules predicted to be capable of interaction with these proteins. The method includes the steps of predicting the configuration of a binding site in said target protein, dividing the binding site into a plurality of grid points, generating a three-dimensional density map of preferred atom-atom contact distributions in the binding site and generating a molecular interaction search template from said three-dimensional density map.

This invention concerns a method for generating lead molecules for interacting with proteins. In particular it relates to a method that identifies the binding sites of proteins, characterises the types of atomic interactions available within those binding sites, and uses this information as a means of identifying lead molecules predicted to be capable of interaction with these proteins.

All references cited in this document are incorporated herein by reference in their entirety.

One of the primary goals in pharmaceutical research remains the discovery of small molecules that will be active in the treatment of disease. Such molecules may cure a disease, or alleviate its symptoms, by interacting with a specific protein and either inhibiting it or increasing its activity.

The inhibition of a protein may be desirable, for example, if the symptoms of a particular disease are caused by the overexpression of that protein. Alternatively, where a protein is crucial to a pathogen, such as a bacterium or a virus, inhibition of the protein may weaken or even destroy the pathogen.

In addition, there are circumstances where the activation of a protein may be desirable, for example, where the symptoms of a disease are caused by a deficiency in an enzyme. Lipoprotein lipase, or the Clearing Factor Lipase activity, is an enzyme that breaks down fat molecules. An example, therefore, of where activation of a protein may be desirable is where there is a deficiency of Clearing Factor Lipase, leading to an accumulation of fats, or lipoproteins, in the blood. The activation of Clearing Factor Lipase by the binding of small molecules (or “agonists”) may reduce clots in the cardiovascular area.

A further example of where the activation of a protein is useful is the activation of adrenoreceptors. Activation with an agonist of a beta 3-adrenoreceptor related to dietary obesity may inhibit weight gain. Alternatively, these agonists may increase the oxygen consumption of the heart and simulate physical exercise by increasing heart rate, blood pressure and the force of contraction of the heart.

Molecules that display some potential for interacting with a target are termed “lead molecules”. By “lead molecule” is meant herein a molecule or molecular fragment that displays some potential for interacting with, or is predicted to interact with, the whole or part of a binding site of a target protein. The binding site of a target protein may contain within it an active site, particularly in the case of enzyme molecules. An active site may be defined as the general region of an enzyme molecule containing the catalytic residues identified with the binding and reaction of substrate(s).

If biochemical experiments indicate that such lead molecules bind to a target protein with micromolar or nanomolar binding constants, then the drug discovery cycle of lead molecule generation, lead molecule optimisation, drug regulatory approval and surveillance, can begin.

Structure-based drug design, sometimes referred to as in silico drug discovery, is becoming increasingly important in this field. Given knowledge of the three-dimensional structure of a target protein and of its binding site, molecular modellers are able to design lead molecules that are predicted to interact with the binding site of a target protein. Lead molecules may either inhibit the protein, for instance by inhibiting the binding of the protein's natural substrate or cofactor to disable the protein's active residues and prevent it from carrying out its normal function, or may activate the protein.

There have been a number of notable successes in this field, such as for inhibitors of HIV protease, thrombin and serine proteases. However, one disadvantage of structure-based drug design is that it requires knowledge of the three-dimensional (3D) structure of the target protein. At present, the 3D structure of a protein may only be deduced reliably using X-ray crystallography. Other methods are available, including NMR spectroscopy and electron crystallography, but these are less reliable.

The experimentally-determined atomic co-ordinates of many proteins (and of any associated molecules, such as cofactors, ligands, metal ions and water molecules) are deposited in the Protein Data Bank (PDB), managed by the Research Collaboratory for Structural Bioinformatics (RCSB) (http://www.rcsb.org/pdb), where they are made publicly available. Currently, the PDB contains over 12,000 protein structures and is expanding rapidly, with approximately 50 protein structures per week being deposited. However, many of the structures are of proteins whose structures have been determined previously under different conditions or bound to different ligands. Nevertheless, the PDB provides a large amount of information about protein structure and how those structures relate to protein function.

Many pharmaceutical companies carry out their own protein structure determinations, using X-ray crystallography, NMR spectroscopy or both, and have their own in-house data banks listing the atomic co-ordinates of proteins in which they are interested. These protein structures are not generally deposited in the PDB.

Often the 3D structure of a particular target protein is not known. In this case, the structure of a closely related protein is usually chosen as the basis for a model of the unknown structure. This is known as a homology model. The closer the relationship between the proteins, the better the homology model is likely to be. Using present technology, reasonable models can be obtained from proteins of known structure whose sequence identity is as low as 35% compared to the target protein.

Where only sparse information on the sequence identity of a target protein is known, other methods for predicting the 3D structure of the protein are often used. The most successful approach is presently that of “threading”, where the structure of the target protein is predicted directly from its amino acid sequence. This method uses a library of unique protein folds that are derived from structures that are deposited in the PDB. The sequence of the target protein is optimally threaded onto each protein fold in turn, allowing for relative insertions and deletions in the loop regions. The different trial threadings are each assigned an “energy” score based on summing the pairwise interactions between the residues in the given threading. The library of folds is ranked in ascending order of energy, with the lowest energy being taken as the most probable match.

Once the 3D structure of a target protein has been obtained, whether experimentally or via homology modelling or threading, the next step is to determine the location of any binding sites on the protein. The location of binding sites on proteins depends largely on the biological function of the protein. For example, enzymes and transport proteins may bind one or more small molecules and/or metals in a cleft in the surface of the protein, whereas the binding site of a DNA-binding protein is a fold that allows it to position itself on the surface of the DNA, possibly in such a manner as to allow recognition of a specific sequence of DNA nucleotide bases. Some proteins perform their biological roles by interaction with other proteins and their binding sites tend to be fairly flat, with a specific pattern of hydrophobic and polar residues on the interface.

For many proteins, the binding site is obvious from the 3D structure. For example, if the structure is a protein-ligand or protein-DNA complex, then the location of an binding site is where the ligand or DNA is bound. However, the ligand may only interact with a very specific region of the binding site and may disguise the importance of other regions of the binding site, which might be useful to consider during lead molecule optimisation.

Alternatively, the identities of the binding site residues may be known from mutation experiments. Here, the binding site must be at the position where mutated residues which abolish activity occur in the structure.

Sometimes, mutation studies may not be available for a target protein but may be known for closely related protein structures and hence the position of the binding site can be inferred on the basis of structural similarity.

However, there are cases where the binding site of a target protein cannot be inferred simply and must be located by analysis of the structure itself. There are a number of methods that aim to do this. Most depend on locating clefts in the surface of the protein or cavities within the protein followed by identification of those features most likely to be associated with the binding site of the protein.

Once a binding site on a target protein has been located, the next step in the drug design process is to design a molecule that will interact with the binding site with high enough affinity to inhibit the function of the protein or to activate the function of the protein. There are a number of computational approaches that have been used. These include methods that search through databases of small molecules, trying to “dock” each small molecule, in turn, into the binding site. The small molecules are usually docked in many different orientations and positions and allow for some flexibility in the molecule being docked. The “energy” of each trial docking is computed, and the ones with the lowest energies are retained. Consequently the docking procedure may produce a list of potential lead molecules that can be tested by pharmaceutical chemists.

Other computational approaches attempt to “build” novel molecules into the binding site. The molecules are often built from fragments, firstly, by locating where particular functional groups are likely to interact with the binding site and, secondly, by linking these fragments together using appropriate linker fragments.

In both approaches, some means is required of evaluating how favourably a molecule, or a molecular fragment, will interact with the binding site. The most accurate and reliable methods, namely those that perform quantum mechanical calculations, are unfeasible for protein structures due to the number of calculations that would be necessary. More commonly used are methods that make use of semi-empirical potential energy functions to model the energy of interactions between pairs of atoms. Well-known potential energy functions are AMBER, CHARMm and GRID. Other methods focus on interaction sites such as locations where a molecule can form a good hydrogen bond with a polar atom of the protein or where the surface is hydrophobic and hence conducive to hydrophobic interactions.

Other methods use purely empirical scoring functions. These methods use observed two or three dimensional distributions of one atom type relative to another, or one atom type relative to a given fragment type (rather than modelling atom-atom or atom-fragment interactions) using equations whose parameters are fitted with data derived from experiments. The observed distributions generally originate from experimentally determined structures of small molecules, proteins or protein-ligand complexes. For example, several methods, such as ISOSTAR (Bruno et al., J. Computer Aided Mol. Des., 11, 525-537, 1997.) and LUDI (Bohm 1992, J. Computer-aided Mol. Design 6, 61-78, 593-606) use empirical rules based on favourable interaction geometries between different atom types and functional groups to compute favourable interaction sites. These rules are derived from the small-molecule structures in the Cambridge Structural Databases (Allen et al. 1979, Acta Crystallog. Sect B, 35, 2331-2339). In contrast, X-SITE (Laskowski et al., 1996, J. Mol. Biol., 259, 175-201) uses empirical rules derived from the Protein Data Bank and, consequently, benefits from the more comprehensive data available there.

The usual result of the above procedures is the generation of one or more lead molecules that are predicted to interact with the target protein.

The next stage is to verify experimentally which of the lead molecules, if any, are genuine contenders as lead molecules, that is, have a high affinity for the target protein. Samples of each lead molecule are either obtained “off the shelf” or are synthesised. Each lead molecule is then tested against the target protein and its binding strength measured. Any that have millimolar, micromolar or, more preferably, nanomolar binding constants are considered to be genuine lead molecules.

These initial results are generally referred back to a molecular modeller for further design refinement in order to improve the binding strength of the lead molecule with respect to a particular target protein. After modification, further synthesis and testing is required. This cycle may iterate many times before a suitable lead molecule is developed and passed to the next stages of the drug development cycle; namely, the lead molecule optimisation stage which incorporates toxicology, oral availability, drug metabolism, pharmacokinetics and, finally, clinical trials.

Of course, the process of drug design is not merely the design of a single lead molecule that can interact with a target protein. Often candidate lead molecules fail at the clinical trial stage, that is after five to six years of research investment, due, for example, to side effects that are caused by the activity of the molecule on proteins other than the target protein. Consequently, a candidate lead molecule needs to be potent against the protein target and also highly specific to that target, rather than simply to members of the same protein family as the target, which may also serve an important biological role in the body that is unconnected with the condition in question. Thus, consideration needs to be given to the specificity of the interactions between the target protein and the candidate lead molecule, and how or whether this specificity varies across the entire protein family. By designing lead molecules to take advantage of specific interactions, they can be made to target just one member of the family. Alternatively, a molecular series can be designed containing a general motif (or scaffold) with a separate region that can be tailored to target any one of the various members of the family specifically.

Although there are numerous computational tools available for the various stages of the structure-based drug design process described above, there is presently no seamless computational system that is easy and comprehensible enough to be used by persons other than expert molecular modellers and computational scientists. Academic or industrial computational tools tend to address specific parts of this problem. For example, the POCKET program (Levitt, D. G. and Banaszak, L. J., J. Mol. Graphics 1992, 10, 229-234), which is a computer graphics method for identifying and displaying protein cavities and their surrounding amino acids, can be used to identify potential binding sites. The GRID potential (Goodford, P. J. 1985, J. Med. Chem. 28,849-857) calculates regions within the binding site that have a high affinity for different types of “probes” using a semi-empirical potential. Thus, it can be used to compute favourable interaction sites for different atoms or functional groups within the binding site of a target protein. The DOCK program (Kuntz, I. D., Blaney, J. M., Oatley, S. J., Langridge, R. & Ferrin, T. E., J. Mol. Biol. 1982, 161, 269-288) is a geometric approach to molecular interactions that will dock every molecule in a database of small molecules into the binding site of a target protein and report on the best hits that it finds. The MSI Ludi program (Bohm, H. J., J. Comput. Aided Mol. Des. 1992, 6, 61-78) is a method for the de novo design of enzyme inhibitiors that can perform fragment searches to identify molecular fragments that will most readily interact with a target enzyme.

Current computational methods to find potential drug lead molecules thus require many different applications to be run in turn. The output from each application must also be processed in preparation for running the next application in the sequence. These methods require considerable computer expertise. Furthermore, file formats must be changed between the different software packages used, which requires a significant input of time. Even though major molecular software houses, such as Tripos and MSI, aim to provide comprehensive and all-inclusive computational tools integrated with sophisticated computer graphics software, these require considerable computer expertise, training and practice.

Consequently, the chemists, medicinal chemists and biologists, who are crucial to the drug design process, tend to be unable to make use of the available computational tools.

There is thus a need for a means of analysing data relating to the binding sites of target proteins and for the prediction of lead molecules capable of interaction with such binding sites in a way that is commonly comprehensible. There is also a need for making the computational process simple enough for all those involved in the drug design process to use, in order to improve communication between the different research groups involved and shorten the research cycle.

Most importantly, despite recent advances in our ability to study protein structure, there presently remains a need for a method of generating a model for a lead molecule designed to interact with a particular target protein, generated from detailed information of the shape and configuration of the binding site and containing information about the optimum constituent atom types and the position that each atom type should adopt within the binding site. Such a model would significantly reduce the length of the drug design process by providing vital information on the types of molecules that are predicted to interact with the target protein. In particular, as these models provide information on the whole of the available binding site, they may provide increased specificity for the target protein.

According to the present invention there is provided a method of generating a molecular interaction search template for a lead molecule predicted to be capable of interacting with a target protein, said method comprising the steps of:

(a) predicting the configuration of a binding site in said target protein;

(b) dividing the binding site into a plurality of grid points;

(c) generating a three-dimensional density map of preferred atom-atom contact distributions in the binding site and

(d) generating the molecular interaction search template from said three-dimensional density map.

The method of the invention generates a molecular interaction search template for a target protein, which can be used to identify lead molecules predicted to interact with the target protein.

By the term “molecular interaction search template” is meant the complementary configuration of the surface of a protein binding site. This takes into account not only shape complementarity but also the chemical interactions that are optimal at each point in the vicinity of the binding site. Thus, in addition to taking spatial considerations into account, this template takes account of sites where a molecule might form strong hydrogen bonds or other electrostatic interactions, or could take advantage of hydrophobic interaction forces.

The molecular interaction search template may also be interpreted as the complementary binding molecular surface required by the optimum ligand/substrate/drug of a given protein. The ligand can be a small molecule (such as a low molecular weight drug substance, including small natural or synthetic organic molecules of up to 2000 Da, preferably 800 Da or less in size), peptide or protein.

To allow a molecular interaction search template to be generated using the method of the invention, a structure should ideally be known for the target protein. At present, the largest depository of protein structures is the PDB database. In addition to this database, many commercial institutions have now generated private databases which contain information relating to protein structures. These structures are of course also suitable for analysis according to the method of the present invention. Furthermore, as will be described in further detail below, in the absence of a fully solved 3D structure, it is often possible to use structures of closely related proteins to derive sufficient information to allow a molecular interaction search template to be generated using the method of the present invention.

The first stage in the method is to predict the configuration of a binding site in the target protein. Where the structure has already been included in the PDB, or in a private database of solved structures, this calculation may already have been carried out. In this case, information on the location and configuration of the binding site of the target protein may simply be accessed in order to perform the method of the present invention.

Where the stage of identifying clefts in the surface of the structure comprises accessing a database where pre-calculated data from structures included in the PDB are stored, a preferred embodiment of the invention uses PDB files read into the “XMAS” format, a format derived from the original PDB files and which contains crucial interpretations of the structure(s) represented by the PDB file, for example relating to protein chains, DNA chains and ligands bound, and with many errors in the original files corrected. The XMAS format is described in co-pending, co-owned PCT patent application No. PCT/GB01/01123, the content of which is incorporated herein in its entirety.

Conventional methods for the location of clefts and cavities in protein structures generally compute the “negative image” of the protein structure, usually by computationally “filling” the empty space around the protein with spheres of variable size. Internal cavities are fairly easy to locate, as they are regions of space that are totally enclosed by protein atoms. Hence any void-filling method using spheres is provided with a natural boundary for the sphere-filling exercise.

Surface clefts, on the other hand, are more difficult to define because of the problem of determining how far the spheres should extend into open space, that is, deciding where the “sea level” lies for that part of the protein. A common solution to this problem is to consider all pairs of atoms in the structure and to locate all the void re-ions between them. This procedure, which restricts voids within the outer limits of the surface of a protein, locates both internal cavities and surface clefts.

The process of binding site identification can be performed for any known structure. Programs already exist that are capable of performing this exercise, such as SiteID (Tripos Inc., 1699 South Hanley Rd., St. Louis, Mo., 63144, USA), VOIDOO (Kleywegt & Jones Acta Cryatallogr. 1994, D50, 178-185), HOLE (Smart, Goodfellow & Wallace Biophys. J. 1993, 65, 2455-2460) and POCKET (Levitt, D. G. and Banaszak, L. J., J. Mol. Graphics 1992, 10, 229-234.) However, although these methods detect internal cavities, most do not detect surface grooves. Furthermore, none of them predicts which void region might be the actual binding site of a target protein.

In a preferred embodiment of the invention, cleft identification, whether precalculated or performed for a specific structure, involves the use of the published SURFNET algorithm (Laskowski, 1995, J. Mol. Graph., 13, 323-330) or, more preferably, an algorithm based on the SURFNET algorithm.

In SURFNET, all possible pairs of atoms in the protein are considered in turn. A sphere is placed midway between the surfaces of the two atoms so that it just touches each. The surface of each atom is taken to be at its van der Waals radius (the radii used being listed in Table 1 below; hydrogen atoms are preferably ignored).

TABLE 1
Atomic van der Waals radii.

| Number | Atom type | Van der Waals radius (Å) |
|---|---|---|
| 1 | N.ar or N.am | 1.65 |
| 2 | N.4 | 1.50 |
| 3 | C.2 | 1.76 |
| 4 | C.3 | 1.87 |
| 5 | O.2 or O.3 | 1.40 |
| 6 | S.3 | 1.85 |

If the radius of the sphere is larger than a threshold value (conveniently any value larger than the largest van der Waals radius used, that is, 1.87 Å, preferably about 4.0 Å), then that sphere is not considered any further. Otherwise, any overlap between the sphere and neighbouring atoms is removed by gradually reducing the radius of the sphere. If the radius drops below a minimum threshold value (conveniently from 1.0 Å to the value of the smallest van der Waals radius used, 1.4 Å, preferably about 1.0 Å), the sphere is also not considered further. Where a sphere survives the overlap tests with all neighbouring atoms, its location and final radius are recorded.

Once all atom pairs in the target protein have been considered in this way, the result is a number of clusters of interpenetrating spheres, both inside the protein and on its surface, corresponding to cavities and clefts. Surfaces may be generated around these sphere clusters to give a solid representation, or a “negative image”, of the voids in the structure of the protein. The surfaces may be generated as 3D density maps using a grid-spacing, as described in Laskowski (1995, loc. cit.) in which a grid spacing of 0.8 Å is used. These surfaces can be viewed using standard molecular graphics packages, for example, QUANTA™ (Molecular Simulations Inc., 9685 Scranton Road San Diego, Calif. 92121) and Sybyl™ (Tripos Inc., 1699 South Hanley Rd., St. Louis, Mo., 63144, USA).

One problem with the published SURFNET algorithm is that this method tends to recognize minor tracks and indentations in the protein surface and identify these areas as potential binding sites. Often these tracks run along the protein surface like rivulets that join separate clefts together, meaning that several distinct cleft regions can fuse into an apparently much larger filamentous region that in fact has little biological significance. Thus, rather than defining binding sites as single, simple regions that are easy to study, this algorithm tends to reveal larger, multiply connected regions joined by channels that are far too narrow to accommodate the binding of any drug molecule. The net result may be that the shape of the binding site often appears to be larger and more complicated than it is in reality.

Accordingly, a preferred embodiment of the method of the present invention uses a modified version of the SURFNET algorithm, which aims to overcome this problem. The modified algorithm proceeds in a similar manner to the original algorithm until the stage where 3D density maps are computed, at which point there is an additional filtration step comprising positioning a sphere of a certain radius (conveniently between 1.6 Å and 2.0 Å, preferably around 1.8 Å) at every grid point in the 3D array. In addition to the grid-point at which it has been placed, each sphere will encompass a plurality of other grid-points, preferably between 50 and 60 grid points, more preferably between 54 and 58 other grid points and most preferably about 56 other grid-points. If all of these grid points belong to one of the “void” regions in the 3D density map, then the sphere is retained. If a single grid point lies outside the void region, the sphere is rejected. In this manner, narrow channels on the protein surface are filtered.

Once all grid points have been considered in this way, the retained spheres are used to generate a 3D density map, as in the original SURFNET algorithm. The density map generated by the improved algorithm is simpler and cleaner, without the misleading filamentous channels.

The next stage is to analyse which cleft or cavity is most likely to be associated with the protein's binding site from the number of clefts identified on the surface of the protein. Most commonly, the binding site is the largest cleft. However, there are other indicators of whether this cleft is the binding site; the surface of a true binding site will tend to be more hydrophobic than other parts of the protein surface. The relative size of the cleft is also an indicator; a cleft that is much larger than any others is almost certainly the binding site, whereas one only marginally larger than any other may not be. If a cleft is located between two domains in the fold of the protein, then it is also more likely to be the primary binding site. Various properties of each cleft should be compiled, including the cleft volume and the accessible surface of all the protein atoms within a certain distance (conveniently around 4.0 Å) of the cleft “surface”. The cleft volume is calculated by summing all the grid-points within the cleft region and converting into Å³ using the grid-spacing.

Many protein structures are solved as complexes with a small molecule bound in their binding site. For these structures, determination of the identity and configuration of the binding site is trivial, in that this is the cleft in which the ligand molecule is found.

Where the solved structure is of the protein alone, the binding site can often be inferred either by reference to related structures in the PDB which include a bound ligand, or by reference to the “SITE” records in the PDB file of the given structure (or of a close relative) which define the binding site residues of the protein. Again, the associated cleft can be taken to be the binding site.

Where none of the above information is available, that is, where there is no ligand bound in the structure, no SITE records included in the PDB file and no closely related structures have been solved, other information may be accessed. For example, the properties of the various clefts in the surface of the protein can help to identify the binding site. For example, it has been shown that where the largest cleft is significantly larger than all others, the cleft is almost always associated with the binding site (Laskowski et al., 1996, Protein Science, 5, 2438-2452). The relative size of the largest cleft may be measured by taking the ratio of its volume to that of the second largest cleft. If this ratio is greater than a threshold value, then the largest cleft can be taken to be the binding site. Advantageously, the threshold value is obtained empirically from the known binding sites of a large number of proteins. Preferably, the threshold value for the ratio is about 1.4. Of course, larger values for the threshold ratio increase the certainty of the assignment of the binding site but reduce the number of cases for which the binding site can be predicted.

Where the ratio is lower than this threshold value, further evidence is generally required. This may be obtained from the cleft properties mentioned above. In this method, a second ratio may be computed for each cleft, which is the ratio \sqrt{A}/³\sqrt{V}, where A is the accessible surface area of all protein atoms within a certain distance (for example 4.0 Å) of the cleft surface and V is the cleft volume. The cleft with the lowest ratio \sqrt{A}/³√V is most likely the binding site, even if it is not the largest, as a low ratio indicates a more enclosed cleft.

With this method, secondary or dual binding sites may be identified, which is particularly relevant where a cofactor is required for activation or inhibition of a target protein. For example, protein kinase requires two binding sites, for ATP and a substrate.

In step b) of the method of the invention, the binding site should be generated as 3D density maps using a grid-spacing of grid points so that the spatial configuration of the binding site may be represented mathematically. Any form of grid-spacing may be used. Preferably, the binding site is divided into grid-points using a three-dimensional density map that incorporates a grid-spacing of between 0.5 and 1.0 Å, more preferably a grid spacing of about 0.8 Å, for example, as described in Laskowski (1995 loc. cit.).

The template generation is performed using a knowledge-based potential that describes favourable atom-atom interactions available within the binding site of the target protein. This potential has been derived Empirically from proteins and protein complexes of known structure and is based on the 3D spatial distributions of atomic contact preferences between different atomic types. One significant advantage of this method is that it uses no assumptions about energy functions. The method uses rules that are based entirely on atomic interactions that have been observed in known protein structures. This means that the rules reflect the way in which atoms interact in real proteins.

In contrast to any of the conventional methods discussed in the literature, the molecule-protein interaction is considered from the point of view of the molecule rather than from that of the target protein itself. This reduces the chance of missing any interactions required by the protein at its binding site.

Once a binding site on a target protein has been identified, either by accessing database records or by mathematical computation, and a grid has been established of this site, the next stage in the method of the invention is the prediction of favourable atomic interactions available within the binding site, involving the generation of a three-dimensional density map of preferred atom-atom contact distributions in the binding site. Preferably, this comprises the steps of:

i) dividing the amino acid residues in the vicinity of the binding site into three-atom fragments;

ii) for a terminal atom of each three atom fragment in the binding site, calculating the preferred atom-atom contact distributions for each one of a plurality of probe atoms, the preferred contact distributions being taken from a database of calculated atom-atom contact distributions; and

iii) retaining contact distributions that fall at grid points within the predicted binding site.

By “database of calculated atom-atom contact distributions” is meant herein a database comprising information relating to regions around protein fragments where particular atom types, such as carboxyl oxygen and main chain nitrogen, may interact favourably with the nearby atoms of the protein. For example, this database may be generated using the X-SITE algorithm, as outlined below.

The steps outlined above for a preferred embodiment of this invention allow predictions to be made of what type of molecule(s) might interact with the protein, with a view to designing a molecule that might potentially act as a drug against the associated disease.

Preferably, the algorithm that the method of the invention uses for discovering the chemical characteristics of the binding site is based on the published “X-SITE” algorithm (Laskowski et al., 1996, J. Mol. Biol., 259, 175-201) or on an algorithm that is based on the X-SITE algorithm. The X-SITE algorithm maps out the regions within the binding site that are favourable for different atom types, that is, it identifies regions where particular atom types, such as carboxyl oxygen and main chain nitrogen, can interact favourably with the nearby atoms of the protein.

The X-SITE algorithm and the improvements discussed herein are based entirely on empirical results and as such make no assumptions about the nature of the forces that exist between atoms. Instead, the algorithm relies entirely on the compilation and use of a database of 3D distributions of the different atom types about different 3-atom fragments. The 3D distributions are taken from known protein structures in the PDB. Not all the PDB structures are used to generate this database, as there are many similar, or even identical, protein structures in the PDB. To use the whole data bank would thus bias the results obtained. Therefore, a set of non-homologous proteins is preferably selected from the PDB and used for compiling the distributions. The distributions can be recompiled as the size of the PDB grows, and the intention is to recompile them at least quarterly.

Full details of the X-SITE algorithm are given in Laskowski et al., 1996 (J. Mol. Biol., 259, 175-201), the contents of which are incorporated in its entirety. For convenience, the general principles of the method are described below.

Each protein is notionally broken up into its constituent, overlapping, 3-atom fragments. The fragments consist of three covalently bonded atoms in a row of the form A-B-C, where the hyphens represent the covalent bonds. There may be additional fragments that overlap these, such as B-C-D, C-D-E and D-C-B. As only contacts to the third atom in the fragment are considered, fragments A-B-C and C-B-A are not equivalent. 3-atom fragments are preferably used as they define a unique frame of reference.

The fragments in a given target protein are transformed onto a common reference frame and all other atoms that make contact with the third atom in the fragment are transformed with them. The reference frame is such that, for example, all three atoms of fragment A-B-C, lie in the x-y, plane with atom C at the origin, B on the negative x-axis, and A with a positive y-value. Only the atoms from the remainder of the protein that are physically in contact with atom C are considered for the accumulation of the distributions. The 3D disposition of each of these, in the new reference frame, is stored and contributes to the 3D distribution of that atom type about that fragment type.

The method of the present invention preferably uses an extended X-SITE algorithm. In this extended version, 20 different atom types (Table 2) and 39 different types of 3-atom fragments are used in the algorithm, giving a total of 20×39 distributions. These atoms differ slightly from those described in the original X-SITE paper.

TABLE 2
Definition of Atom type

| | | | Chemical |
|---|---|---|---|
| | Probe name | Atom types in PDB | symbol |
| 1 | Backbone N | NH1 N | N.am |
| 2 | Charged N | NH3 | N.4 |
| 3 | Uncharged N | NH2 NH1S NC2 | N.ar |
| 4 | Backbone O | O | O.2 |
| 5 | Carboxyl O | OC OS | O.co2 |
| 6 | Hydroxyl O | OH1 HOH | O.3 |
| 7 | Backbone C | CH1E CH2G | C.2 |
| 8 | Backbone CA | C | C.3 |
| 9 | Aromatic C | CR1E CR1W CRHH CR1H | C.ar |
| | | CH0 CF CY CW C5 C5W CY2 | |
| 10 | Aliphatic C | CH1S CH2E CH2P CH3E | C.3 |
| 11 | All sulphurs | SH1E SM | S.3 |
| 12 | Chlorine | C1 | C1 |
| 13 | Fluorine | F | F |
| 14 | Sulphonate sulphur | S2 | S.2 |
| 15 | Amine | N4 | N.3 |
| 16 | CN nitrogen | N5 | N.1 |
| 17 | NO₂ nitrogen | N6 | N.2 |
| 18 | Sulphonate oxygen | O4 | O.2 |
| 19 | NO₂ oxygen | O6 | O.2 |
| 20 | CN carbon | C5N | C.1 |

The distributions generated by X-SITE are stored as arrays of grid-points in 3D space, centred at the origin. Each grid-point has a value that is proportional to the density of the given atom type at that point in the reference frame.

Preferably, not all contacts are stored, as very many are artefacts of the local secondary structure of the protein and so would heavily bias the distributions with these local interactions. Thus, contacts between main chain atoms which are so dominant and numerous in protein structures, and so heavily influenced by secondary structure, are only stored if they correspond to “long-distance” main chain to main chain contacts (that is, where the atoms involved come from amino acid residues that are at least three residues apart in the linear sequence of the protein). Similarly, any contacts involving sidechain atoms are ignored if they involve residues that are adjacent in the linear sequence of the protein.

In the original X-SITE algorithm, an atom-atom “contact” was defined as occurring where the centre-to-centre distance between the two atoms was less than the sum of their van der Waals radii (see Table 1), plus 1.0 Å. The preferred X-SITE algorithm for use in the present invention additionally includes a cut-off distance to simplify the normalisation of the different atom and fragment distributions, whereby the atoms are deemed in contact if their centre-to-centre distance exceeds a threshold value. This value is conveniently taken as about 6.0 Å, which is the maximum atom-atom contact distance, but may be less than 6.0 Å.

The data set of protein structures used in compiling the distributions ideally consists of protein chains taken from structures solved by X-ray crystallography to a resolution of 2.0 Å or better, and with an R-factor no lower than 20%. By “R-factor” is meant herein the ratio of the diffraction amplitudes observed experimentally to those calculated for a hypothetical crystal of a model protein structure.

To ensure a non-redundant (and hence unbiased) dataset, the chains should be selected such that no two chains share a sequence identity of greater than 25%, preferably no greater than 20%. At the time of writing, this gives 365 protein chains from the PDB database but as the number of known protein structures increases, the distributions will be periodically regenerated with progressively more data.

The method of this invention preferably incorporates a number of modifications to the original X-SITE algorithm. The first significant preferred modification is the addition of extra atom types. The original algorithm considered only those atoms found in the 20 amino acid residues that constitute protein molecules. Consequently it omitted several atom types that are not found in proteins but which are significant in drug design (for example, chlorine, fluorine, oxygen and nitrogen from nitro-groups, nitrogen from cyano-groups, sulphur from sulphonyl-groups, and so on). The extended algorithm includes these extra atom types by identifying them in ligand molecules bound to proteins in the PDB and deriving their 3D distributions around the fragments already described. The advantage of this extension of the algorithm is that the presence of these extra atom types is not overlooked, even though the occurrence of these atom types is less than those for atoms found in proteins. Currently, the distributions for these 9 extra atom types (types 13-20 in Table 2) come from 334 structures of protein-ligand complexes taken from the PDB.

A second preferred modification concerns the normalisation of the 3D distributions. The original algorithm was rather complicated and was unsuccessful in giving useful predictions for the extra atom types just described. The relative paucity of their contact data adversely affected the normalisation procedure. In the modified algorithm, the density of observations at each grid-point is converted into a probability. The probabilities are computed separately for each atom type and it is assumed that every atom is equally likely to be present, irrespective of the relative amounts of data obtained for each from the data set. The database is, of course, heavily biased towards those atoms found in amino acids and, in particular, towards the atoms which form the main chains of proteins.

For example, where there are n_{frag} 3-atom fragments, a 3D grid containing the observed contacts for each of the n_{atype} probe atom types can be constructed. There are, therefore, n_{frag}×n_{atype} possible grids.

Each grid is of size n_{grid}=n_{g}×n_{g}×n_{g}, where ng is the number of grid-points along the x-, y- and z-axes (conveniently set at n_{g}=21, with a grid-spacing of 0.8 Å).

For any atom type, aⱼ, the distribution of that atom around each of the n_{frag} fragments is contained within n_{frag} grids and the total number of observations of atom type aⱼ across all these grids is Nₐⱼ. Most of these observations of atom aⱼ in one grid will be dependent to some extent on observations in other grids. In the protein structures from which the data are drawn, a given atom of type aⱼ will be in contact with several fragments and so contribute to several grids. However, for the purposes of normalization, all observations are assumed to be independent.

Each grid will contain a different number of observations of atom type aⱼ because of differences in the propensities of an atom type to be at a particular grid point with respect to the different fragments and because of different “available volumes” within which atom type aⱼ can make contact with fragment type fₖ. The latter is a function of the radius of the “contact” atom in fragment type fₖ and of the context within which the fragment is located in the protein structure. For example, for a main chain fragment such as N—Cα—C, where the grids define observed long-distance contacts to the carbonyl C, no atom can possibly be located at the position of the carbonyl oxygen covalently bonded to the C atom. Hence, a large part of the grid is occluded by the O atom. Similarly, the N of the next residue, which is attached to the C, rules out another region of the available grid points.

The accessible region for fragment type fₖ is computed by superposing all 3D grids from all n_{atype} different atom types. The grid-points containing no observations are taken to be inaccessible and are excluded from further consideration.

Thus, for each fragment type, a number of accessible grid-points, n_{kaccess}, are identified and a total across all the fragment types is calculated:

$${n}_{access} = {\sum }_{k = 1}^{{}^{n} frag} {n}_{kaccess}$$

If it is assumed that atom type aⱼ has an equal probability of being observed at any of the n_{access} accessible grid-points, then the probability density function (hereafter, “pdf”) across all the grid-points is A(gᵢ|aⱼ), given by:

$$A ( gi \vert aj ) = \frac{1}{{n}_{access}} for i = 1 , 2 , … , {n}_{access}$$

where gᵢ is grid-point i. This is the a priori distribution of the pdf for atom type aⱼ.

The a priori distribution is converted into an estimated pdf for atom type aⱼ using the observations across the n_{frag} 3D grids for that atom type. The approach follows that of Sippl for treating sparse data sets (Sippl, 1990, J. Mol. Biol., 213, 859-883) as expanded for multidimensional frequency tables by Andrej \check{S}ali (PhD thesis 1991, University of London).

If the relative observed frequencies of atom type aⱼ across all the accessible grid-points are W(gᵢ|aⱼ), then the pdf for the atom type is given by

P(gᵢ|aⱼ,fₖ)=ω_{j,1}A(gᵢ|aⱼ)+ω_{j,2}W(gᵢ|aⱼ)

where ω_{j,1} and ω_{j,2} are weighting parameters given by

$${ω}_{j , 1} = \frac{1}{1 + {N}_{a j} / ( σ {n}_{access} )}$$

ω_{j,2}=1−ω_{j,1}

where σ is a parameter that determines the relative contributions of the measured and a priori distributions. When the average number of observations per grid-point is σ, the two contributions are equal.

Once derived, the distributions can be applied to the identified binding site of any target protein structure as described in Laskowski (1996) loc. cit. The binding site of the protein is examined in order to predict favourable interaction regions for each of the different “probe” atom types for which distributions have been derived.

Accordingly, the residues of the binding site are, notionally, broken up into their constituent 3-atom fragments and the appropriate 3D atom-fragment distributions are taken from the database of pre-calculated data and superposed onto each fragment, giving a set of overlapping densities within the binding site itself. Regions of high density for the distribution of a particular atom type correspond to favourable interaction regions for that atom. The net result is a mosaic of favourable regions for each of the different probe atom types.

The next stage in the method of the invention is the generation of a molecular interaction search template for a lead molecule predicted to be capable of interaction with a target protein, and significantly extends work previously reported in Laskowski, 1995 (loc. cit.) and Laskowski et al., 1996 (loc. cit.).

This stage in the method comprises the step of calculating which probe atom types have the highest probability of occupying each grid point.

Preferably, this stage of the method comprises the steps of:

i) calculating probability density functions that determine, at each grid point, which probe atom type has the highest probability of occupying that point; and

ii) overlaying the probability density functions generated in step i) to give a unified three-dimensional grid map of preferred occupancies for each grid point in the binding site.

In this preferred embodiment, the prediction generated in the previous stage of the method for each probe atom is stored as a 3D grid of probability densities encompassing the region of the binding site. The higher the value at a given grid-point, the higher is the likelihood of finding that type of atom at that location.

In step i) above, the probability densities for each probe atom at each grid-point in each 3D grid are compared, meaning that the atom type with the highest probability of occupying each point in the binding site may be determined. This is done by generating a probability density map for each probe atom, which is similar to the original probability density map for that atom but with only the values at those grid-points where the probe atom scored the highest being retained, all other grid-points being set to zero. The net result is a new set of 3D grid maps, one map per probe atom, each map holding only those regions where a particular atom scored higher than the others.

Of course, because of the nature of the grid, the constituent points are situated more closely together than the positions that atoms may occupy naturally. Accordingly, the method of the invention may include a method of averaging the results at neighbouring positions in the grid in order to make sense of the preferred contact distributions and allow the conversion of these data into a template of preferred molecular occupancies.

The molecular interaction search template generated according to the method of the present invention may have a large surface area. Screening for suitable components to generate a lead molecule based on this large molecular interaction search template may include protein-protein interactions in addition to databases of small molecules. The surface area for small molecule binding is usually in the range 300-1000 Å², while for protein-protein interactions a surface area of greater than 1000 Å² is typical.

In one aspect of the present invention, the method may comprise an additional step involving the generation of a pharmacophore. By “pharmacophore” is meant herein the spatial arrangement of features contained in a lead molecule that is predicted to interact with the whole or part of a target protein.

In a preferred embodiment of this aspect of the present invention, the generation of a pharmacophore comprises the steps of:

i) generating favourable interaction regions for each of the probe atoms; and

ii) overlaying the favourable interaction regions for all probe atoms.

In a particularly preferred embodiment of the present invention, these favourable interaction regions are generated by creating, for each probe atom, a set of spheres from the molecular interaction search template, such that a sphere represents an averaged favourable interaction region for a particular probe atom. For example, a method of approximation involves placing a sphere at each non-zero grid-point and progressively increasing the size of the sphere up to a maximum threshold radius (conveniently, this is taken to be around 30 Å, but any radius above about 4.0 Å will work equally effectively). At each step of growth the number of zero and non-zero grid-points within the sphere are counted and the percentage of non-zero grid points calculated. When the percentage drops below a certain cut-off value, preferably between 60% and 90%, more preferably around 75%, the growth of the sphere is stopped.

Once all non-zero grid-points have been processed, various clusters of interpenetrating spheres of differing radii remain. These may be filtered so as to leave only a small number of the most significant and representative spheres. This filtering process involves first sorting all the spheres into descending order of size. The spheres are then considered in turn, from the second largest to the smallest. For each, if any larger spheres are found to overlap with a smaller sphere over a threshold value (such as, for example between 40 and 60%, preferably around 50%, of the radius of the smaller sphere), then the smaller sphere is deleted.

In this manner, a set of spheres of different atom types is generated that represents the favourable interaction regions for each atom probe. Each sphere is described by its radius, the x-, y- and z-co-ordinates of its centre, and by the type of atom that it represents.

The step of overlaying the favourable interaction regions may then be performed. This step may involve, for example, overlaying the probability density functions generated in step i) above to give a unified three-dimensional grid map of preferred occupancies for each grid point in the binding site.

In the preferred embodiment involving the generation of sets of spheres that represent the favourable interaction regions for each atom probe, these sets of spheres are simply overlaid. In this manner, the pharmacophore is produced, which is a model of the areas within the binding site of the target protein that are predicted as most favourable for each of the different probe atoms.

In a preferred embodiment, each sphere in the set of overlaid spheres does not overlap with any other sphere in the set of overlaid spheres.

In a further embodiment of the present invention, the method may additionally comprise the step of fitting a lead molecule that is predicted to be capable of interaction with the target protein into the molecular interaction search template. Preferably, the lead molecule is fitted into the molecular interaction search template by placing a plurality of molecular fragments from a database of small molecular fragments at each of the grid points within the binding site. The positions of the atoms within the fragments may be compared with the probability densities for the most favourable atom probes within the molecular interaction search template.

Alternatively, or additionally, the method may additionally comprise the step of fitting a lead molecule that is predicted to be capable of interaction with the target protein into the pharmacophore. Preferably, the lead molecule is fitted into the pharmacophore by placing a plurality of molecular fragments from a database of small molecular fragments at each of the grid points within the binding site. The positions of the atoms within the fragments may be compared with the favourable interaction regions within the pharmacophore.

Where a lead molecule is fitted into the molecular interaction search template or into the pharmacophore, a database of small molecular fragments, for example, a commercially or corporately available database or a built-in database of small molecular fragments or combinatorial chemical libraries, may be used. Typically, the molecules being screened will be small, having a molecular mass in the range 300-800. Preferably, such a database includes fragments comprising the most common organic functional groups and which represent the common building blocks used in chemistry. These fragments may include, for example, those listed in Table 3. Preferably, these fragments correspond to the most drug-like elements in drug molecules (Bemis and Murcko, 1999, J. Med. Chem., 42, 5095-5099; Bemis and Murcko, 1996, J. Med. Chem., 39, 2887-2893). Such virtual screening in silico promotes the fast discovery of potential drug candidates.

TABLE 3
Fragment Examples

| Fragment Name | Structure |
|---|---|
| Phenyl | 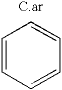 |
| Carboxyl | 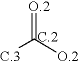 |
| Carbonyl | 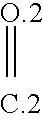 |
| Amide | 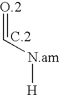 |

Each fragment is defined in terms of its constituent atoms, that is its “probe type” (as in Table 2), and their x-, y- and z-coordinates. Each fragment in the database may be then placed at every grid-point within the binding site and subjected to a number of rotations (for example 100 rotations). At each rotation, a score is calculated, for example using the appropriate X-SITE predictions for the atom types that the fragment contains.

As one example, a carbonyl fragment may be considered. This group, containing a carbonyl oxygen (type 4 in Table 2) and a carbonyl carbon (type 7 in Table 2), is placed at each grid-point, initially in the orientation defined by its original co-ordinates. The positions of the carbon and oxygen atoms are compared with the probability densities for those atom probes at those positions in the molecular interaction search template, respectively, and a score is deduced relating to the degree to which the current location and orientation for the carbonyl fragment is favourable as a whole.

A score is calculated for each of the rotations and the orientation giving the highest score at this grid-point is stored. This procedure is repeated for the same fragment at the next grid-point.

Once all grid-points within the binding site have been covered, the highest-scoring locations, and associated orientations, for the fragment are retained.

This procedure should be repeated for all the fragments in the database. In this manner, information is obtained on which fragments appear to have favourable interaction regions within the binding site and, in particular, exactly where and at which orientation these interactions are most favourable.

The fragment distributions within the binding site are useful for providing chemists with ideas on the sorts of lead molecules that are likely to interact at these positions and can be used for searching small molecule databases for candidate lead molecules. They can also be the starting point for the design of combi-chem libraries.

The combination of different fragments together with the distance constraints between them can be used to generate sub-structural search templates. The program will preferably output at least three types of search queries: for the Tripos™ Unity package, the MDL ISISBase™ and the Daylight database (Daylight Chemical Information Systems Inc., 27401 Los Altos, Suite #360, Mission Viejo, Calif. 92691). However, as the skilled reader will appreciate, the output may easily be modified, as desired.

In this manner, it is possible to pick out lead molecules that best match the query constraints, namely, the required fragments and the distance constraints between them. A lead molecule identified using the molecular interaction search template or the pharmacophore is predicted to be capable of interacting with a target protein binding site with high affinity. Such a molecule is a “lead” in the sense that until the molecule itself is generated and used in conjunction with the protein in biochemical binding studies, its ability to interact with the target protein remains only a potential ability. However, the method of the invention is sufficiently rigorous and meticulous to enable a high level of confidence to be placed in the predictions that are generated. Preferably, such lead molecules interact with a protein of interest with at least micromolar affinity, preferably millimolar affinity, more preferably nanomolar or greater affinity. In addition, the lead molecules should ideally exhibit specificity for the target protein. By “specificity” is meant that the lead molecule interacts strongly only with the target protein, preferably not interacting with any degree of affinity to other proteins.

In a further embodiment of the invention, the method of the first aspect of the invention may incorporate the step of generating a fingerprint that stores or represents the chemical and physical properties of the molecular interaction search template or of the pharmacophore. The inclusion of this step facilitates fast searching of molecular interaction search templates or pharmacophores in a database.

Fingerprinting (or Keyed Searching) describes an approach whereby the available information on a collection of structures or molecules is synthesised into one or more binary keys, thereby providing a means of rapidly screening the collection on the basis of key attributes. As long as the binary “fingerprint” keys contain information pertinent to the most commonly-used search criteria, pre-screening using these keys can rapidly reduce the number of structures of interest to a manageable level, at which stage more rigorous (or time-consuming) selection criteria may be applied. This approach would seem to be highly suited to filtering, for example, the binding site information generated for proteins, and even the protein molecules themselves.

The steps of such an approach are outlined briefly below.

Firstly, a list of the key structure attributes that will be represented in the binary “fingerprint” is generated. These might include both qualitative and quantitative information (see below). A Binding Site Key might include the following information:

Selected Atom types present;

Selected Centre types present;

Selected X-SITE distribution;

Selected H-Bond component;

Selected Residue types present;

Pharmacophores present;

Surface Area;

Included Volume;

Conformation of selected residues or features; and

Geometry of selected residues or features.

It is important that, as far as possible, the attributes selected for use in binary keys may be determined precisely for all structures being fingerprinted. Binary keys, by their nature, may only convey “present” or “absent” information and there is little capacity for “unknown”. Binary keys are intended to provide fairly low level information for rapid screening purposes only. For example, a number of the residues listed above may be inaccessible in the binding site. However, the key would only show whether they were or were not present. Attributes such as accessibility may be provided by using a more detailed key, but would generally be the subject of a secondary analysis, after Key screening had reduced the number of structures of interest to manageable proportions. Crude quantitative data may also be included in keys.

These attributes may then be categorised into groups on the basis of a common functionality, where possible. Although this is not strictly necessary, this does allow the screening process to be optimised later. Binary keys are generally (although not invariably) held in short or long unsigned integers and therefore hold a maximum of 16 or 32 bits of information. Again, screening may be optimised by using multiple keys, for example separate Atom/Centre Key, Residue Key, Physical Attributes Key, etc.

A mechanism may be provided for holding the separate keys in conjunction with the data that they represent. The binary Keys may be held within a single database-type file for rapid screening, either with or without the base data. Alternatively, the keys may be entered as fields in a commercial database that allows searching to be performed. The mechanism adopted depends on the amount and type of data being screened, as well as local software and hardware requirements.

A preliminary set of Keys is then generated and a search performed on them. Developing the most efficient set of binary Keys for a given set of structures requires a high degree of optimisation.

The method of the present invention may be performed for a collection of proteins and the results compiled into a database. Such a database forms a further aspect of the present invention and contains information relating to molecular interaction search templates and/or to pharmacophores and/or to lead molecules for a plurality of target protein structures.

Advantageously, a database according to this aspect of the invention may contain data relating to all proteins of known structure and may optionally include proteins whose structure has not been solved using the conventional techniques of X-ray crystallography, NMR spectroscopy and electron crystallography, but which have been predicted in silico, either from first principles (for example, by sequence comparison and threading techniques) or by analogy with closely related proteins whose structures have been solved. Ideally, the database of this aspect of the invention will therefore be updated as frequently as possible.

In addition to containing information relating to molecular interaction search templates and/or to pharmacophores and/or to lead molecules for a plurality of target protein structures, a database system according to the invention may incorporate a plurality of computer programs for processing protein and lead molecule structure information, and a database of results entries containing results records generated by the application of the computer programs to the molecular interaction search templates and/or to the pharmacophores and/or to the lead molecules.

Such databases described above will be of use for a number of scientific applications. For example, the database may be consulted in order to discover details of the shape and the chemical constitution of an optimum molecule for interacting with the binding site of a particular protein of interest. The database may hold the molecular interaction search templates or pharmacophores for some or all proteins in a particular consensus family. Examination of the information concerning the different consensus families stored in the database may lead to the discovery of common features, in addition to features that may be included in a lead molecule to impart specificity to a particular protein within the consensus family. The database may also be used in training neural networks to derive a set of rules that can be used as the basis for the validation of target proteins.

Due to the complexity and number of calculations that the method of the invention necessitates, it is envisaged that the methods and databases of the invention will only be implementable using computational means. The output of the method may preferably be suitable for manipulation in unrelated computer programs, for example for searching in the Tripos™ Unity package (UNITY4.1 & SYBYL 6.6, Tripos Inc., 1699 South Hanley Rd., St. Louis, Mo., 63144, USA) or in DOCK (Kuntz et al., 1982, J. Mol. Biol., 161, 269-288). Both packages can perform searches of small molecule databases and pick out the molecules that best match the molecular interaction search template and/or the pharmacophore to identify lead molecules for the target protein, both in terms of the shape of the binding site and the chemical properties of the atoms at different points within the binding site.

Furthermore, the computer-implemented method may be configured for use as a net-based program, which may be accessed when looking for viable lead molecules for a target protein. The database may be stored locally and accessed by an intranet or local server, or may be selected from a remote site, for example over the internet.

The method may also be linked to one or more other bioinformatics resources, for example to the “Biopendium™” database that is described in co-owned, co-pending PCT patent application No. PCT/GB01/01105, the contents of which are hereby incorporated in their entirety. This database system comprises pre-calculated analyses of all known protein structures in the PDB, and may include any other structures solved in-house by a subscribing company.

If the target protein has any related structures, then the analyses on these may be considered for comparative purposes. Such comparisons may be particularly crucial in the design of lead molecules that are highly specific against the target and which are less specific against other members of the same consensus family as the target. The starting point for any such analysis is a structural alignment of the entire family of proteins. A structural alignment differs from a sequence alignment in that it aligns protein structures on the basis of their overall fold rather than only on sequence homology. It can include very distantly related proteins that share low sequence homology, sometimes even lower than 10%, yet which have a very similar overall structure. For example, pre-calculated structural alignments may be taken from the Biopendium™ database, which uses the SSAP algorithm of Orengo and Taylor (Orengo & Taylor, 1996, Methods Enzymol., 266, 617-635) to perform structural alignments.

The superposed structures may be displayed graphically, as can their binding sites and favourable X-SITE interaction predictions. Together this can highlight the similarities and differences in the binding sites and so provide valuable ideas during the drug design process.

According to a further aspect of the invention there is provided a computer apparatus adapted to compile a database or a database system as described in any one of the embodiments described above. Such a computer apparatus may preferably contain the following elements:

a processor means comprising:

a memory means adapted for storing data relating to molecular structures;

first computer software stored in said computer memory adapted to predict the configuration of a protein binding site;

second computer software stored in said computer memory adapted to generate a three-dimensional map of preferred atom-atom contact distributions in the binding site;

third computer software stored in said computer memory adapted to generate a molecular search interaction template for said protein binding site; optionally fourth computer software stored in said computer memory adapted to generate a pharmacophore for said target protein binding site; and further optionally

fifth computer software adapted to fit a lead molecule predicted to be capable of interacting with said binding site into the molecular interaction search template and/or into the pharmacophore.

The invention also provides a computer-based system for generating a lead molecule predicted to be capable of interacting with a target protein, said system involving the steps of:

a) inputting information relating to the identity or structure of the target protein;

b) interrogating a database or database system as described above; and

c) outputting one or more lead molecules predicted to interact with the target protein.

The invention also provides a computer-based system for generating a lead molecule predicted to be capable of interacting with a target protein, comprising the steps of:

a) accessing a database or database system of the type described above;

b) inputting information relating to the identity or structure of a target protein into the database or database system;

c) interrogating the database or database system to identify lead molecules predicted to be capable of interaction with the target protein; and

d) outputting one or more lead molecules predicted to be capable of interaction with the target protein.

Such computer systems as described above may preferably contain the following elements:

a central processing unit;

an input device for inputting requests;

an output device;

a memory;

at least one bus connecting the central processing unit, the memory, the input device and the output device;

the memory storing a module that is configured so that, upon receiving a request to generate a molecular interaction search template for a target protein and/or a pharmacophore for a target protein and/or the structure of a lead molecule capable of interacting with a target protein, it performs the steps listed in any one of the methods of the invention that are described above.

Any one of the methods described above may include an additional step of synthesising one or more of the lead molecules generated by the method. Optionally, the method may also comprise a step of analysing the lead molecule to assess its binding affinity for the target protein binding site and thus its potential suitability as a candidate drug molecule.

According to a still further aspect of the invention, there is provided a computer program product for use in conjunction with a computer, said computer program comprising a computer readable storage medium and a computer program mechanism embedded therein, the computer program mechanism comprising a module that is configured so that, upon receiving a request to generate a molecular interaction search template for a target protein and/or a pharmacophore for a target protein and/or the structure of a lead molecule capable of interacting with a target protein, it performs any one of the methods of the invention described above.

A specific embodiment of the present invention is now described, by way of example only, with reference to the accompanying drawings in which:

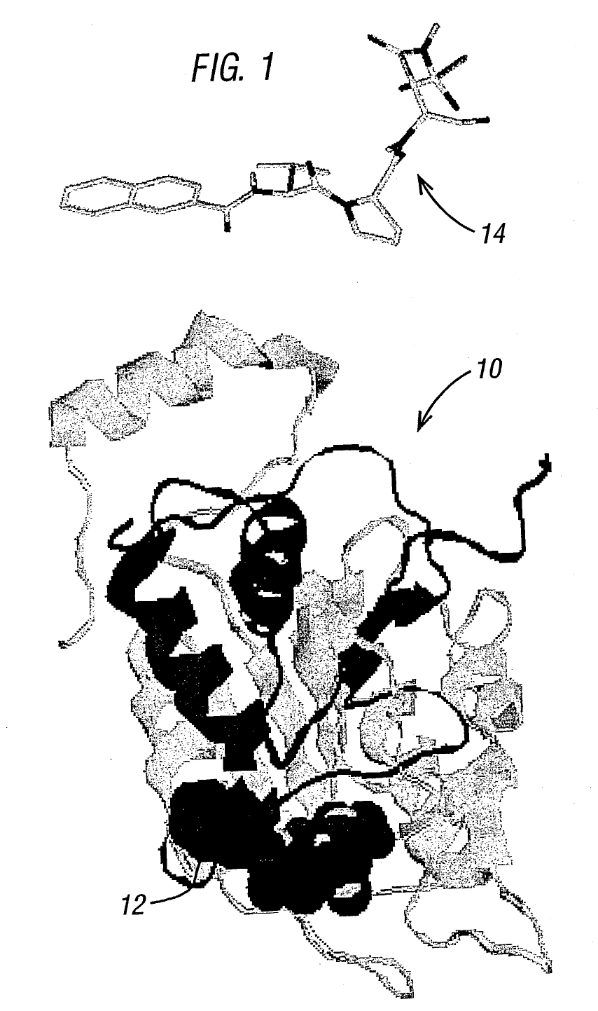

FIG. 1 is a representation of a three-dimensional structure of interleukin-1 beta converting enzyme and a co-crystallised ligand;

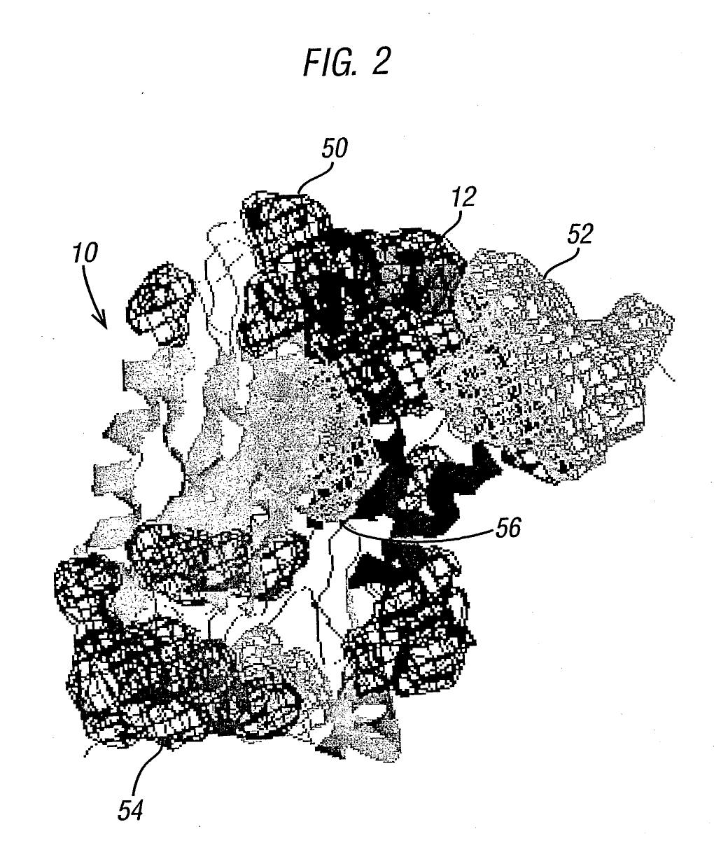

FIG. 2 is a representation of the three-dimensional structure of interleukin-1 beta converting enzyme on which gap regions of the enzyme have been superimposed;

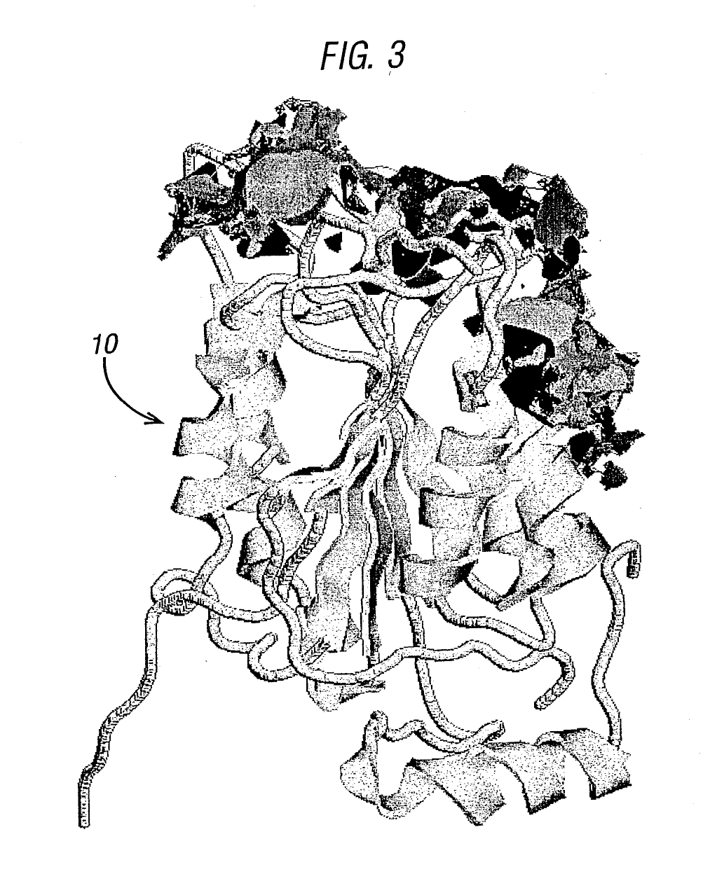

FIG. 3 is a representation of the three-dimensional structure of interleukin-1 beta converting enzyme in which a representation is shown of the three-dimensional probability density map for a plurality of probe atoms in the binding site of the enzyme;

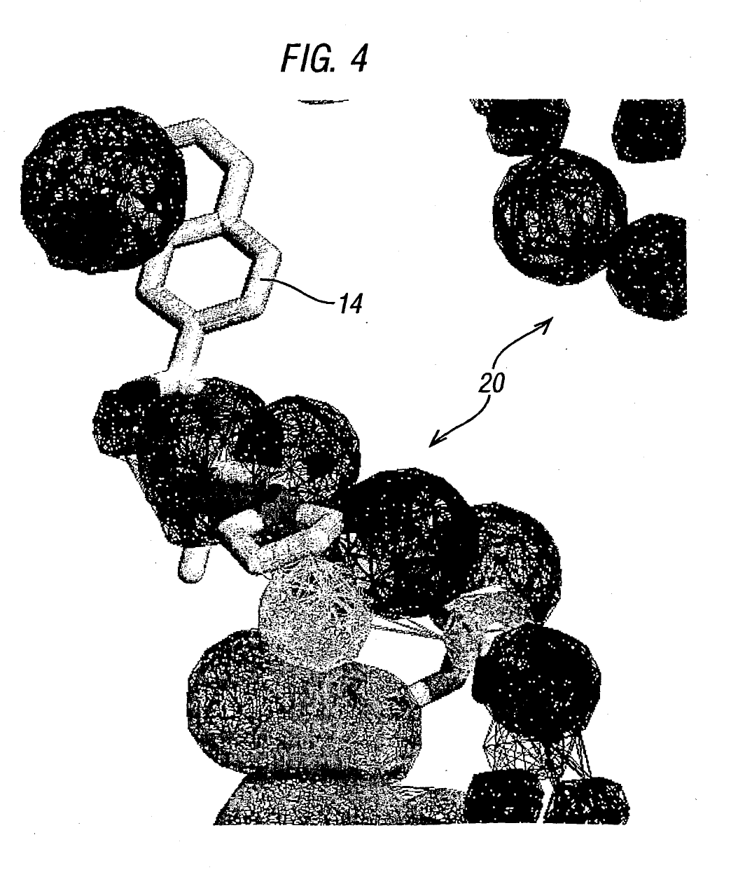

FIG. 4 shows part of a pharmacophore displayed alongside a portion of the co-crystallised ligand of interleukin-1 beta converting enzyme;

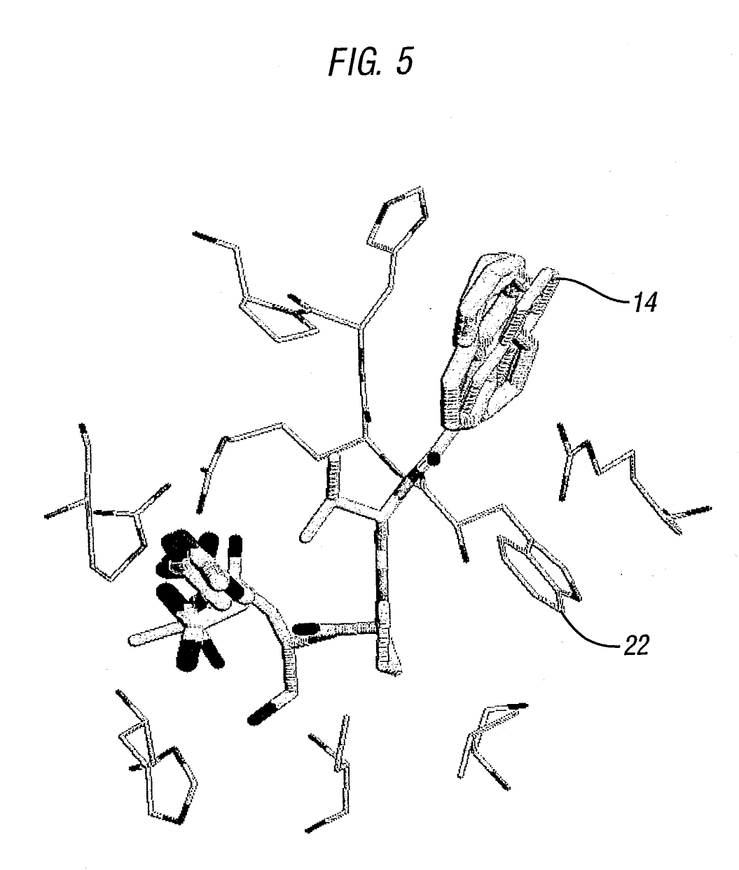

FIG. 5 shows molecular fragments predicted to be capable of interaction with the binding site of interleukin-l beta converting enzyme. The co-crystallised ligand of interleukin-1 beta converting enzyme is also shown; and

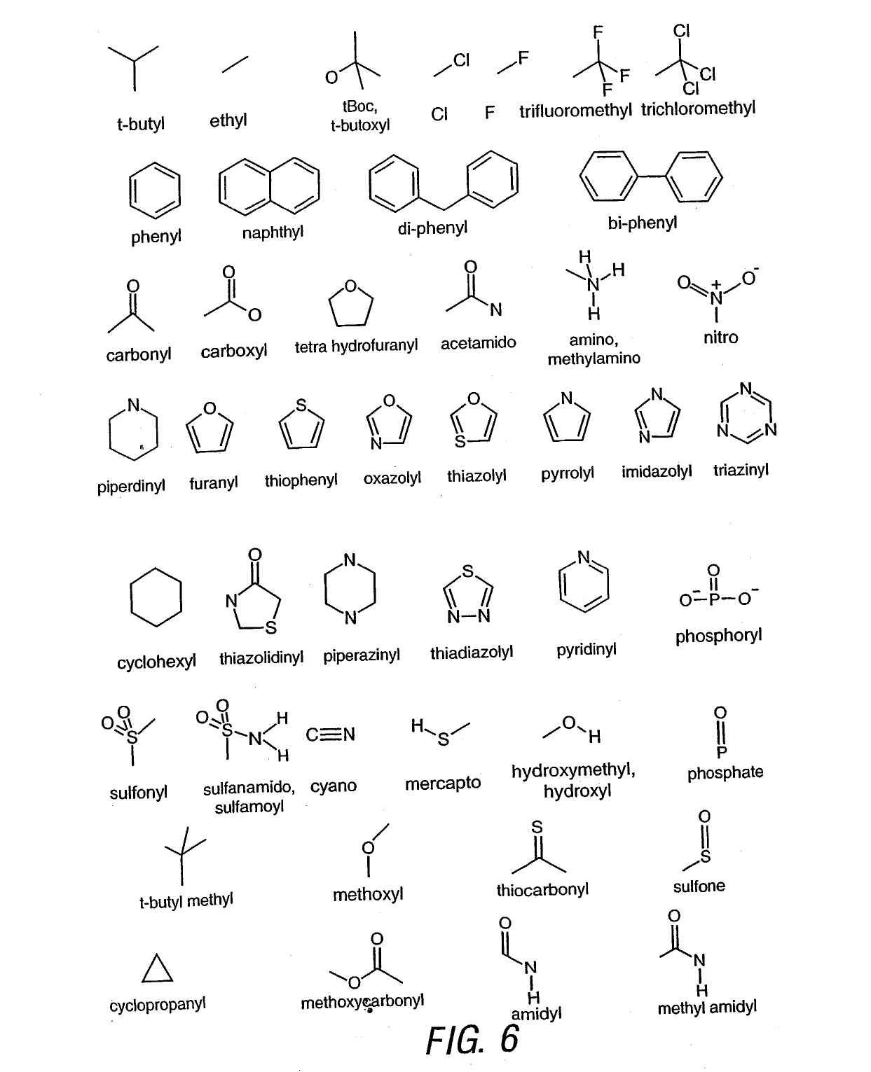

FIG. 6 shows the fragments in the database available for searching.

### EXAMPLE 1

Identification of Lead Molecules for Interleukin-1 Beta Converting Enzyme

With reference to FIG. 1 to FIG. 5, the method of the present invention has been applied to the interleukin-1 beta converting enzyme. A representation of the three-dimensional structure of this enzyme 10 is shown in FIG. 1. The structure of this enzyme is found in the PDB database, under PDB code 1 bmq, having a resolution of 2.50 Å and an R-factor of 0.233. A space-filling model of the structure of the co-crystallised ligand 12 of interleukin-1 beta converting enzyme is shown. Also shown is a representation of the molecular structure 14 of the co-crystallised ligand.

Using an algorithm based on the SURFNET algorithm as described above, the clefts and cavities in the structure of interleukin-1 beta converting enzyme were located and defined. FIG. 2 shows four void regions located in the structure of interleukin-1 beta converting enzyme. In the working version of this method, separate void regions are displayed in different colours for ease of identification. Here, these regions are indicated by reference numerals. The following table summarises the details of the void regions shown in FIG. 2:

| Void region | Volume (Å³) | Reference numeral |
|---|---|---|
| 1 | 2900 | 50 |
| 2 | 1712 | 52 |
| 3 | 1626 | 54 |
| 4 | 712 | 56 |

The binding site was selected from among the four void regions identified above using the criteria for binding site selection described above. In this case, void 1 was selected as the binding site.

As shown in FIG. 2, all atoms of the co-crystallised ligand 12 of interleukin-1 beta converting enzyme 10 fall within this binding site, thereby providing validation for the binding site selection procedure used in the method of the present invention.

The next stage is the generation of three-dimensional probability density maps of preferred atom-atom contacts for each of a plurality of probe atoms within the identified binding site in interleukin-1 beta converting enzyme. FIG. 3 shows a representation of the three-dimensional probability density map of preferred atom-atom contacts for backbone N, charged N, aromatic N, backbone O, carboxyl O, hydroxy O, backbone C, backbone alpha C, aromatic C, aliphatic C and all sulphurs. Although it is not possible to use a colour representation here, it should be understood that in the working version of this method, different colours are used to represent the different groups present.

In the three-dimensional density map, it is possible to select individual probe atoms in order to see the probability density map for a particular atom type. It is also possible to deselect atom probes where the presence of a particular atom type is not desired. For example, in FIG. 3, fluorine, chlorine, CN nitrogen, NO₂ nitrogen and sulphonate oxygen are not selected. All other atoms are shown.

The molecular interaction search template of the binding site of the protein is stored electronically in the memory of a computer apparatus.

The molecular interaction search template was then used to generate a pharmacophore according to the method of the present invention. With reference to FIG. 4, the pharmacophore comprises a set of non-overlapping spheres 20. Each sphere represents an averaged favourable interaction region for a particular probe atom. In the working version of this embodiment of the present invention, each probe atom type is assigned a different colour and each of the possible constituent probe atoms, as listed above, may be selected or deselected. In FIG. 4, fluorine, chlorine, CN nitrogen, NO₂ nitrogen and sulphonate oxygen are not selected when viewing the pharmacophore. In this Figure, part of the pharmacophore is shown overlaid with the co-crystallised ligand 14 of the interleukin-1 beta converting enzyme.

A lead molecule predicted to be capable of binding to interleukin-l beta converting enzyme is identified by placing a plurality of molecular fragments from a built-in database of small molecular fragments into the binding site and comparing the positions of the atoms within the fragments with favourable interaction regions within the pharmacophore. As shown in FIG. 5, the molecular fragments 22 identified to be components of the lead molecule are overlaid with the co-crystallised ligand 14 of interleukin-1 beta converting enzyme. As can be seen from this Figure, there are a number of regions where the predicted molecular fragments and the co-crystallised ligand concur.

A large number of molecular fragments may be tested in this manner. A list of molecular fragments available in the built-in database of this embodiment are shown in FIG. 6.

It will, of course, be understood that the present invention has been described above purely by way of example and that modifications of detail can be made within the scope and spirit of the invention.

1. A method of generating a molecular interaction search template for a lead molecule predicted to be capable of interaction with a target protein, said method comprising the steps of:
a) predicting the configuration of a binding site in said target protein;
b) dividing the binding site into a plurality of grid points;
c) generating a three-dimensional density map of preferred atom-atom contact distributions in the binding site; and
d) generating the molecular interaction search template from said three-dimensional density map.

2. A method according to claim 1, wherein in step a), the configuration of said protein binding site is predicted using the SURFNET algorithm (Laskowski, 1995, J. Mol. Graph., 13, 323-330) or an algorithm based on the SURFNET algorithm:

3. A method according to claim 2, additionally comprising the steps of positioning a theoretical sphere at every grid point in the three-dimensional array identified as forming the binding site, and assessing whether every grid point that is encompassed by said sphere falls within said binding site, wherein spheres are only retained where all grid points encompassed by said sphere fall within said binding site.

4. A method according to claim 3, wherein each sphere has a radius of 1.6 Å to 2.0 Å, preferably 1.8 Å.

5. A method according to claim 4, wherein each sphere encompasses from 51 to 61 grid points, preferably 55 to 59 grid points, more preferably 57.

6. A method according to claim 1, wherein in step a), the configuration of the binding site is predicted using information contained within a database of protein structures.

7. A method according to any one of the preceding claims, wherein in step b), the binding site is divided into grid-points using a three-dimensional density map that incorporates a grid-spacing of between 0.5 and 1.0 Å, preferably around 0.8 Å.

8. A method according to any one of the preceding claims, wherein step c) comprises the steps of:
i) dividing the amino acid residues in the vicinity of the binding site into three-atom fragments;
ii) for a terminal atom of each three atom fragment in the binding site, calculating the preferred atom-atom contact distributions for each one of a plurality of probe atoms, said preferred contact distributions being taken from a database of calculated atom-atom contact distributions; and
iii) retaining contact distributions that fall at grid points within the predicted binding site.

9. A method according to any one of the preceding claims, wherein in step c), the three-dimensional density map of preferred atom-atom contact distributions is generated using the X-SITE algorithm (Laskowski et al., 1996, J. Mol. Biol., 259, 175-201) or an algorithm that is based on the X-SITE algorithm.

10. A method according to claim 9, wherein said X-SITE algorithm is extended by including the three-dimensional distributions of the atoms chlorine, fluorine, sulphonate sulphur or oxygen, amine nitrogen, CN nitrogen or carbon and NO₂ nitrogen or oxygen.

11. A method according to any one of the preceding claims, wherein step d) comprises the step of calculating which probe atom type has the highest probability of occupying each grid point.

12. A method according to claim 11, wherein step d) comprises the steps of:
i) calculating probability density functions that determine, at each grid point, which probe atom type has the highest probability of occupying that point; and
ii) overlaying the probability density functions generated in step i) to give a unified three-dimensional grid map of preferred occupancies for each grid point in the binding site.

13. A method according to claim 12, wherein step d)i) comprises the separate steps of comparing said probability density functions for each probe atom at each of the grid points, determining which probe atom has the highest probability of being present at each grid point in the binding site and creating a probability density map for each probe atom, said map indicating the probability density only at those grid points where the probe atom scored the highest, all other grid points being zero.

14. A method according to any one of the preceding claims, additionally comprising the step e) of generating a pharmacophore from the molecular interaction search template.

15. A method according to claim 14, wherein said step of generating a pharmacophore comprises the steps of:
i) generating favourable interaction regions for each of said probe atoms; and
ii) overlaying the favourable interaction regions for all probe atoms.

16. A method according to claim 15, wherein said favourable interaction regions are generated by creating for each probe atom a set of spheres from the molecular interaction search template, such that a sphere represents an averaged favourable interaction region for a particular probe atom.

17. A method according to claim 16, wherein said each sphere in said overlaid set of spheres does not overlap with any other sphere in said overlaid set of spheres.

18. A method according to any one of claims 1 to 13, additionally comprising the step of fitting a lead molecule predicted to be capable of interaction with the target protein into said molecular interaction search template.

19. A method according to claim 18, wherein said lead molecule is fitted into said molecular interaction search template by placing a plurality of molecular fragments from a database of small molecular fragments at each of the grid points within said binding site and comparing the positions of the atoms within said fragments with said probability densities for the most favourable atom probes within said molecular search interaction template.

20. A method according to any one of claims 14 to 17, additionally comprising the step of fitting a lead molecule capable of interaction with the target protein into said pharmacophore.

21. A method according to claim 20, wherein said lead molecule is fitted into said pharmacophore by placing a plurality of molecular fragments from a database of small molecular fragments at each of the grid points within said binding site and comparing the positions of the atoms within said fragments with said probability densities for the most favourable atom probes within said pharmacophore.

22. A method according to any one of the preceding claims, additionally comprising the step of generating a fingerprint that represents the chemical and physical properties of the molecular interaction search template and/or of the pharmacophore, such that fast electronic searching of a plurality of molecular interaction search templates and/or of a plurality of pharmacophores is facilitated.

23. A database containing information relating to molecular interaction search templates and/or to pharmacophores and/or to predicted lead molecules for a plurality of target proteins, said database being generated by performing a method according to any one of the preceding claims.

24. A database system comprising:
a database containing information relating to molecular interaction search templates and/or to pharmacophores and/or to predicted lead molecules for a plurality of model target proteins;
a plurality of computer programs for processing said information; and
a database of results entries containing results records generated by the application of the computer programs to the molecular interaction search templates and/or to the pharmacophores and/or to the predicted lead molecules.

25. A computer apparatus adapted to compile a database according to claim 23 or a database system according to claim 24; or to use a method according to any one of claims 1-22.

26. A computer apparatus for compiling a database containing information relating to molecular interaction search templates and/or to pharmacophores and/or to predicted lead molecules for a plurality of model target proteins, said apparatus comprising:
a processor means comprising:
a memory means adapted for storing data relating to molecular structures;
first computer software stored in said computer memory adapted to predict the configuration of a target protein binding site;
second computer software stored in said computer memory adapted to generate a three-dimensional map of preferred atom-atom contact distributions in the binding site;
third computer software stored in said computer memory adapted to generate a molecular interaction search template for said target protein binding site; and
optionally
fourth computer software stored in said computer memory adapted to generate a pharmacophore for said target protein binding site; and
optionally
fifth computer software adapted to fit a lead molecule, predicted to interact with said binding site, into the molecular interaction search template and/or into the pharmacophore.

27. A computer-based system for generating the structure of a predicted lead molecule capable of interaction with a target protein, said system involving the steps of:
a) inputting information relating to the identity or structure of said target protein;
b) interrogating a database according to claim 23 to identify lead molecules predicted to be capable of interacting with said target protein; and
c) outputting one or more structures of lead molecules predicted to be capable of interaction with said target protein.

28. A computer-based system for generating a predicted molecule capable of interacting with a target protein, said system comprising the steps of:
a) accessing a database according to claim 23;
b) inputting information relating to the identity or structure of said target protein into said database;
c) interrogating said database to identify lead molecules predicted to be capable of interaction with said target protein; and
d) outputting one or more structures of lead molecules predicted to be capable of interaction with said target protein.

29. A computer system for generating a molecular interaction search template for a target protein and/or a pharmacophore for a target protein and/or the structure of a lead molecule capable of interacting with a target protein, said system comprising:
a central processing unit;
an input device for inputting requests;
an output device;
a memory;
at least one bus connecting the central processing unit, the memory, the input device and the output device;
wherein the memory stores a module that is configured so that, upon receiving a request to generate a molecular interaction search template for a target protein and/or a pharmacophore for a target protein and/or the structure of a lead molecule capable of interacting with a target protein, it performs the steps listed in the methods of any one of claims 1-22.

30. A computer-based method for generating a lead molecule predicted to be capable of interacting with a target protein of interest, comprising the steps of:
a) accessing the database of claim 23, at a remote site,
b) inputting into said database information relating to the identity or structure of said target protein;
c) interrogating said database to identify lead molecules predicted to interact with said target protein, and
d) outputting the structure of lead molecules predicted to be capable of interacting with said target protein in order of predicted affinity and/or specificity for the target protein.

31. A method according to any one of claims 1-22 or 30, or a system according to any one of claims 27-29, additionally comprising a step of synthesising one or more lead molecules.

32. A method according to claim 31, additionally comprising a step of analysing the synthesised lead molecule to assess its binding affinity for the target protein binding site and thus its potential suitability as a candidate drug molecule.

33. A computer program product for use in conjunction with a computer, said computer program comprising a computer readable storage medium and a computer program mechanism embedded therein, the computer program mechanism comprising a module that is configured so that upon receiving a request to generate a molecular interaction search template for a target protein and/or a pharmacophore for a target protein and/or the structure of a lead molecule capable of interacting with a target protein, it performs a method as recited in any one of claims 1-22.
